(19) United States
(12) Patent Application Publication
(10) Pub. No.: US 2011/0319741 A1
(43) Pub. Date: Dec. 29, 2011

(54) Adapting radiotherapy treatment plans

(75) Inventor: Kevin Brown, West Sussex (GB)

(73) Assignee: Elekta AB (publ), Stockholm (SE)

(21) Appl. No.: 12/821,660

(22) Filed: Jun. 23, 2010

(51) Int. Cl. A61B 5/05

(52) U.S. Cl. 600/407

(57) ABSTRACT

Embodiments of the present invention provide a method of pre-approving a range of hypothetical spatial variations of the target whilst an initial treatment plan is generated. This allows the treatment plan to be later adapted to account for spatial variations of the target region falling within those pre-approved ranges, without going through time-consuming steps of quality assurance on the adapted plan.

### FIELD OF THE INVENTION

The present invention relates to radiotherapy, and particularly relates to a method and apparatus for adapting a radiotherapy treatment plan.

### BACKGROUND ART

Modern radiotherapy techniques make use of images of the target region to plan treatment delivery. A planning scan of the target area is taken via computed tomography (CT) or magnetic resonance imaging (MRI), for example, and a treatment plan is constructed.

The generation of a treatment plan is a complicated process that requires significant computing power and resources.

The anatomical structures in and surrounding the target are identified in the planning scan, and a number of clinical objectives specified. For example, the target for radiotherapy treatment (i.e. a tumour) may be set a lower limit for the accumulated radiation dose which must be exceeded, such that the treatment process is adequate to damage or destroy the cancerous tissue. Sensitive structures, known as organs at risk (OARs), may be set an upper limit which must not be exceeded, to avoid causing unwanted damage. There may also be a general criterion that the radiative dose applied to healthy tissue (i.e. any tissue apart from the target) should be minimized.

Generation of the treatment plan must also take into consideration the geometrical constraints of the radiotherapy system in which it will eventually be implemented. Such systems usually employ a source of radiation mounted on a gantry. The radiation source generates a beam of radiation which is shaped and directed by a collimator such as a multi-leaf collimator (MLC). In many modern systems, the combination of radiation source and collimator are rotated around the patient. In this context, therefore, there are a number of limitations which must be applied to the treatment plan generation. For example, the dimensions of the collimator, the maximum speed with which the gantry and the collimator leaves can move (as well as their inertia) and the power of the radiation source all affect the treatment plans which it is possible to implement. Additionally it should be appreciated that the large numbers of parameters that are required to define the treatment plan represent large ‘degrees of freedom’. This means that there are, in general, a large number of diverse treatment plans that can satisfactorily meet the clinical objectives.

The treatment plan so generated is therefore a computer model of the direction and intensity of radiation that is to be applied to a target in a patient. It is a mathematical solution to the clinical objections and constraints input to the treatment plan generator. However, there are reasons why the actual dose delivered may be different to the computer model, for example a deficiency in the way the computer model handles difficult anatomical situations. Therefore, before the plan can be implemented and radiation delivered to the patient, it is important that quality assurance (QA) is conducted to ensure that the actual dose distribution meets the necessary criteria. Once radiation is delivered, any damage caused to the patient because of a faulty treatment plan cannot be undone.

Such quality assurance often takes the form of a practice run in which a patient “phantom” is placed in the radiotherapy system and the treatment plan initiated. The radiation dose applied to the phantom can then be measured to see if it corresponds to the dose as calculated in the treatment plan. If the calculated and measured dose distributions match, the treatment plan is approved for use with the patient.

Alternative methods of quality assurance may be employed. For example, a higher degree of confidence may be placed in treatment plans where similar treatments have been performed before using the same system and the same operating staff. In those cases, one or more probes may be used to measure the radiation dose at one or more points in the treatment area. If the dose at those points matches the calculated dose, there is a high degree of confidence that the treatment plan will cause the system to operate correctly and the plan can be approved.

However, in general the process of quality assurance can take a long period of time, particularly if the initial treatment plan is not approved and a further plan is required.

More recently, the ability to take images of the patient immediately prior to each treatment fraction (whilst the patient is in the treatment position) has allowed radiotherapists to adapt to any changes in the position or shape of the target region that may occur between fractions. If the target has moved, the existing treatment plan can (in theory) be adapted to compensate for this target movement before the treatment is started.

Because the re-imaging and plan adaptation occur with the patient in the treatment position (otherwise known as “online”), there is insufficient time for the plan adaptations to go back through another QA cycle. Also because the patient is in the treatment position it is not possible to deliver the adapted plan to a QA phantom. Whilst the new, adapted plan is created using the same criteria as the original plan, and should therefore be perfectly safe to implement, the system lacks the reassurance given from putting a plan through a QA cycle. Given the large number of degrees of freedom in generating a new plan, it is entirely possible that the parameters defining the new plan may bear no relation to the parameters defining the old plan.

### SUMMARY OF THE INVENTION

The aim of the present invention is to provide the radiotherapist with what is effectively a form of online QA, giving them greater confidence in the proposed adapted plan made in light of target movement.

It has been shown that for many target movements a simple plan adaptation using only minor changes in some of the rotational and translational plan parameters can have dramatically beneficial effects to the dose distribution, and takes minimal time to implement (Rijkhorst et al, Int. J. Radiation Oncology Biol. Phys., Vol 69, No. 5, pp. 1608-1617, 2007). What we propose is that instead of the dose distributions of these possible variations being considered at the time of treatment, they are instead calculated at the same time as the initial treatment plan is being created, before the patient undergoes their first treatment fraction. In this way, the system not only plans the optimum treatment plan for the positions and orientations of the patient's target region and OARS, etc. in the planning scan, but will also calculate the maximum translations, rotations, and possible deformations that the target area can undergo such that a correspondingly adapted treatment plan still meets the clinical objectives. This done, when a new image of the patient is taken prior to the start of a treatment fraction, and an adapted plan is suggested to the radiotherapist, the system already knows if this adaptation falls within the acceptable boundaries as defined by the treatment planning system in the initial, quality assured treatment plan, and can advise the radiotherapist that implementing said suggested changes will result in an acceptable dose distribution being given to the patient. In effect, the adjustment is “pre-quality assured”.

The sampling of the possible adaptations that can be made may be systematic i.e. testing all possible combinations of the inputs to the algorithm, or based on a suitable statistical sampling technique, examples of which are well known in the art as verification methods.

The same algorithm is used to adapt the plan both within the treatment planning system and in the treatment delivery system. Therefore the effect of the adaptations that will be performed using actual measurements have already been characterised and assured for quality.

With the addition of deformational adaptation the new plan can be even more accurate, although this adds increasing complexity to the problem which adds a commensurate amount of time to the processing period.

### BRIEF DESCRIPTION OF THE DRAWINGS

An embodiment of the present invention will now be described by way of example, with reference to the accompanying figures in which;

FIG. 1a is a flowchart of a method according to embodiments of the present invention, performed prior to a course of radiotherapy treatment; and

FIG. 1b is a flowchart of a method according to embodiments of the present invention, performed prior to individual fractions of radiotherapy treatment.

### DETAILED DESCRIPTION OF THE EMBODIMENTS

As described above, the present invention relates to a method of providing quality assurance for a treatment plan that is initially generated prior to the start of a course of radiotherapy treatment, but then later adapted just prior to each fraction of treatment, for example to take account of movement or deformation of the target region since the initial plan was established. The method is therefore split into two stages: a first, “planning” stage, performed prior to the course of treatment beginning; and a second, “adaptation” stage that is performed just prior to an individual fraction of treatment. This second stage may be repeated prior to each fraction of treatment, or a subset of those fractions (for example if significant movement of the target region is not anticipated on a fraction-by-fraction basis). At the lower limit, the second stage may be performed only once during the course of treatment.

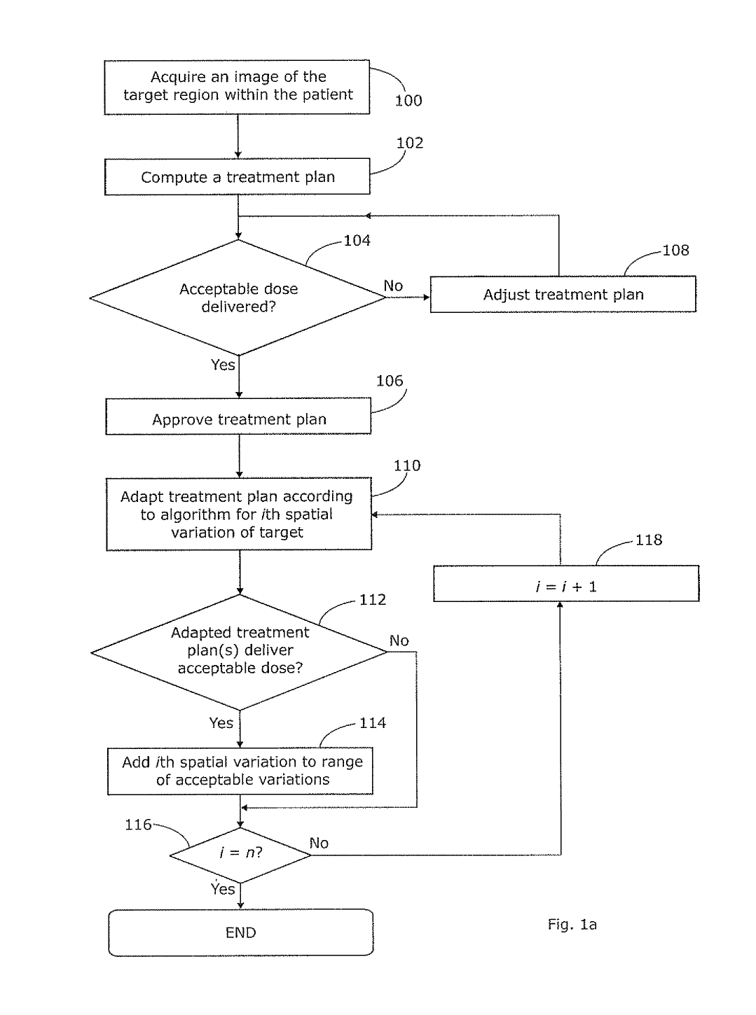

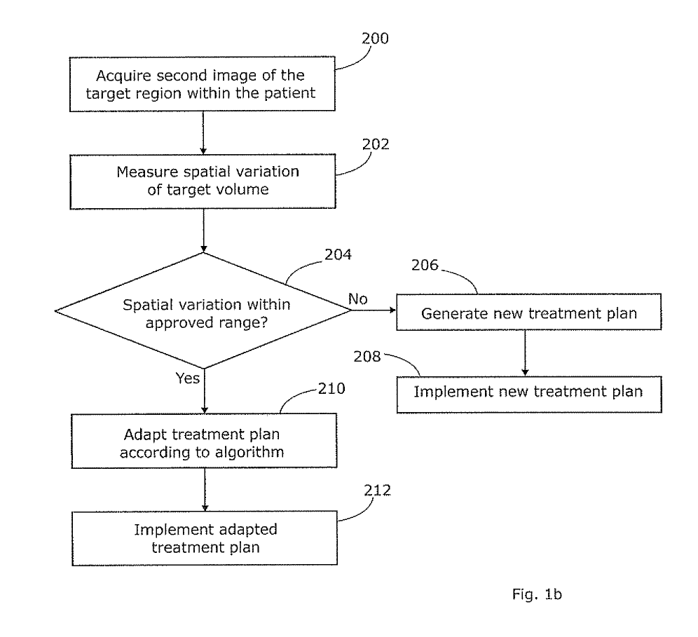

FIG. 1a is a flowchart of the method according to embodiments of the present invention, to be performed prior to the course of treatment beginning, i.e. the first, or planning stage.

The method begins in step 100, in which imaging data is acquired of the target region within the patient. Various methods may be employed to gather the data, and will be familiar to those skilled in the art without further description here. Examples of such methods include magnetic resonance imaging (MRI) and computed tomography (CT).

The imaging data shows the target itself (i.e. a tumour), and will usually also include various nearby anatomical structures and tissue, which may (or will) be unavoidably irradiated as part of the treatment.

In step 102, a treatment plan is generated on the basis of the image obtained in the preceding step.

This process uses as one of its inputs the imaging data, obtained in the preceding step, of the region that will (or may) be irradiated, which has been segmented by manual or automated processes (or some mix of the two) in order to indicate whether a specific part of the image such as a voxel (i.e. a three-dimensional pixel) is either not part of the patient (i.e. free space), part of the tumour or other target to be irradiated, a non-sensitive healthy part of the patient, or a sensitive healthy part of the patient. Target regions are allocated a clinical objective which typically includes a minimum dose that is to be delivered, determined by the clinical outcome that is desired. Sensitive regions (such as certain organs) are also allocated a clinical objective which may be a maximum dose that must not be exceeded, else irreparable damage may be caused to the patient.

In addition, the automated process is provided with details of the constraints imposed on the treatment process by the apparatus that is being used. For example, radiotherapy is often delivered by a linear accelerator-based system, which produces a beam of high-energy x-rays and directs this toward a patient. The patient typically lies on a couch or patient support, and the beam is directed toward the patient from an offset location. During treatment, the beam source is rotated around the patient while keeping the beam directed toward the target point. The result is that the target remains in the beam for most of the time, but areas immediately around the target are only irradiated briefly by the beam during part of its rotation. This enables the dose to the tumour to be maximised whilst the dose to surrounding healthy tissue is reduced.

In addition, the cross-section of the beam can be varied by way of a range of types of collimator, such as the so-called “multi-leaf collimator” (MLC) illustrated in EP 0,314,214. These can be adjusted during treatment so as to create a beam whose cross-section varies dynamically as it rotates around the patient.

Other aspects of the radiotherapy apparatus can also be varied during treatment, such as the speed of rotation of the source and the dose rate. Thus, there are a large number of variables offered by the apparatus in order to tailor the radiation dose that is delivered to the patient. Each of these variables has limits due to the physical shape of an object, its maximum speed, maximum power output, etc.

These various factors and constraints are then expressed as mathematical functions, which enable the process to be automated as a constrained optimisation problem.

In step 104, the generated treatment plan undergoes quality assurance to see if it delivers an acceptable dose to the patient (i.e. whether the delivered dose corresponds closely to the dose calculated as part of the treatment plan). For example, the treatment plan may actually deliver an unacceptably low radiation dose to the target, or an unacceptably high dose to a sensitive structure.

Quality assurance may be carried out in a number of ways, as described above. These include the use of a phantom to replicate the patient and receive the radiation delivered by the treatment plan, or one or more individual measurement probes positioned to measure the radiation at specific locations.

If the radiation dose is acceptable, the treatment plan is approved (step 106). If the radiation dose is not acceptable (for whatever reason), the treatment plan must be adjusted in step 108 by the medical physicist until it complies with the requirements. This process is well described in the literature.

Once a treatment plan has been approved, according to embodiments of the present invention, the method proceeds to find a number of hypothetical spatial variations of the target region which—once the treatment plan has been suitable adjusted—also lead to acceptable treatment plans.

For example, suppose there are n spatial variations which are to be considered, where n is an integer greater than or equal to one. These n spatial variations are broken down into different types of variation (i.e. translation of the target from its starting point, rotation of the target from its starting orientation, deformation of the target from its starting shape, and scaling of the target from its starting size), as well as different magnitudes of variation for each of those variation types (i.e. rotation by 1, 2, 3 or 4 degrees; translation by 1, 2, 3 or 4 mm, etc).

In one embodiment, the only types of spatial variation considered are rotation and translation of the target. It has been shown in the art that these spatial variations can be adequately compensated for by suitable adjustment of the treatment plan (see Rijkhorst E-J et al, “Strategy for online correction of rotational organ motion for intensity-modulated radiotherapy of prostate cancer”, 2007 Int. J. Radiation Oncology Biol. Phys., vol 69 pp 1608-1617). In addition, limiting the possible variations to these two types greatly simplifies the process of quality assurance and treatment plan adaptation.

In one embodiment, the n spatial variations represent a systematic list of all possible combinations of different variation types and different variation magnitudes. For example, the n variations may include positive and negative rotation of the target about an axis in one-degree increments, translation of the target along a set of perpendicular axes in one-millimetre increments, any number of possible deformations of the target shape and scaling of the target size, as well as all possible combinations of these variations. Of course, such an embodiment would take a significant amount of computing resources to complete.

In other embodiments, this workload may be reduced by taking a stochastic sequence of spatial variations, i.e. a plurality of possible variation combinations drawn in a more random manner from the list of possible variations listed above. That is, by a statistical sampling technique, known in the art as a verification method, the number of variations which need to be tested can be reduced.

So, in step 110, the treatment plan is adapted according to an algorithm in order to account for an ith hypothetical spatial variation, or combination of spatial variations, of the target, where i is an integer in the range 0≦i≦n. The algorithm that is applied in order to adapt the treatment plan will be described in greater detail below.

In step 112, the same spatial variation is applied to the target and then this adapted treatment plan is evaluated in order to see if the radiation dose it delivers to the hypothetically adapted target is acceptable. This determination may be made using the same quality assurance methods described above with respect to step 104 or by a simple calculation of the dose. If the radiation dose is acceptable, then the ith hypothetical spatial variation is added to a list of acceptable spatial variations in step 114, and the method proceeds to step 116. If the radiation dose is not acceptable, the method proceeds directly to step 116.

Step 116 is a determination if i=n, i.e. if the list of hypothetical spatial variations has been completed. If not, the method proceeds to step 118, in which i is incremented by 1, and then loops back to step 110 for an evaluation of the next hypothetical spatial variation.

If the list of hypothetical spatial variations has been evaluated, this stage of the process is complete. The user is left with a list of possible target spatial variations which, once a suitable adaptation has been applied to the initial treatment plan, lead to adapted treatment plans that deliver acceptable radiation doses. This list can then be used to develop ranges of spatial variation that also lead to adapted treatment plans delivering acceptable radiation doses. For example, say one of the spatial variations considered acceptable was a target rotation of 1°. That result may be extrapolated to deduce that any rotation between 0° and 1° would be acceptable. This is possible as the adaptation problem is a continuous mathematical function and the adjustments are small. There are no jumps or discontinuities which may lead to a 0.5° rotation being unacceptable if both 0° and 1° rotations are acceptable. Similar deductions can be made from the other measured spatial variations.

FIG. 1b is a flowchart of the second stage of the method according to embodiments of the present invention, the adaptation stage. As stated above, this stage may typically be performed before an individual fraction of treatment, in order to update the treatment plan to account for interfraction movement or other alteration of the target region.

In step 200, imaging data is acquired of the target region of the patient. As described above with respect to step 100, various methods (e.g. MRI, CT, etc) may be employed to gather the data, and will be familiar to those skilled in the art without further description.

In step 202, the spatial variation of the target is measured, relative to its position and orientation in the image obtained in step 100. The spatial variation may include any change in the shape, position or orientation of the target (e.g. one or more of rotation, translation, deformation or scaling).

In step 204, the measured spatial variation is compared with the range of pre-approved spatial variations generated using the method described with respect to FIG. 1a. If the variation does not fall within that range, the method proceeds to step 206 in which a new treatment plan is generated, and step 208 in which that treatment plan is implemented. In some cases, if the spatial variation of the target is particularly severe, the course of treatment may have to be interrupted in order to generate an adequate treatment plan.

If the spatial variation is within the pre-approved range of variations, the treatment plan approved in step 106 is adapted according to an algorithm to account for the spatial variation (step 210). It is important that the algorithm employed in this step is the same algorithm used to adapt the treatment plan in step 110. Thus, the user can be confident that the treatment plan which is now to be implemented has the same properties as the treatment plan that was pre-approved in step 114, that is, the user can be confident that the treatment plan delivers an acceptable radiation dose.

In step 212, the treatment plan is implemented in the radiotherapy system, and radiation delivered to the patient.

Plan Adaptation Algorithm

Various algorithms for adapting the treatment plan may be considered for use with the present invention.

At one extreme, each possible spatial variation may be taken into account in the adapted plan. For example, the target region may be translated a distance from its starting location, and/or be rotated around an axis by several degrees. These variations have implications for the angles of rotation of the multi-leaf collimator (MLC) and the radiation source, as well as the position of the patient within the radiotherapy system. However, the target region may also change shape (i.e. deform) and reduce in size (i.e. scale). These variations have implications for the positions of the MLC leaves, as ideally the radiation beam should accurately conform to the shape of the target region, to minimize damage to surrounding healthy tissue.

Typically an MLC may include 80 pairs of opposing leaves, each individual leaf able to take a position within a continuous range of travel across the MLC housing. It is not difficult to see that taking account of deformation and scaling of the target greatly increases the complexity of the adaptation process. This has consequences not only for the amount of time required to adapt the treatment plan, but also the confidence that the algorithm will arrive at the same adapted treatment plans in steps 110 and 210. With so many degrees of freedom, there may be several alternative solutions to the problem and the user needs to be confident that the same solution was reached in both the planning stage and the adaptation stage.

Thus, in an embodiment of the present invention, only translation and rotation of the target are taken into account when updating the treatment plan in steps 110 and 210. The measured spatial variation may include deformation or scaling of the target region, but these variations are discounted in the adaptation of the treatment plan.

In such an embodiment, the treatment plan is adapted by applying only a minor perturbation that corresponds to the spatial variation of the target. Take a first example, in which the target undergoes a rotation of 3°. To account for this, the collimator may be similarly rotated by 3° in the adapted treatment plan, with other parameters remaining largely unchanged. In a second example, the target may have been translated a distance of 5 mm. To account for this, the patient can be repositioned to place the target correctly relative to of the radiotherapy system. The Rijkhorst et al 2007 paper referred to above, the contents of which are incorporated herein by reference, has shown that such perturbations are successful in tracking and delivering updated treatment plans to targets such as a cancerous prostate. That paper describes methods by which a treatment plan can be adapted to take account of rotational movement of the target.

Nonetheless, if the adapted treatment plan is required to take into account deformations of the target, it may do so by employing a suitable algorithm. Such algorithms include a cubic spline deformation model, for example.

Embodiments of the present invention therefore provide a method of pre-approving a range of hypothetical spatial variations of the target whilst the initial treatment plan is generated. This allows the treatment plan to be later adapted to account for spatial variations of the target region falling within those pre-approved ranges, without going through time-consuming steps of quality assurance on the adapted plan.

It will of course be understood that many variations may be made to the above-described embodiment without departing from the scope of the present invention.

Although the present invention has been described with reference to preferred embodiments, workers skilled in the art will recognize that changes may be made in form and detail without departing from the spirit and scope of the invention.

1. A method of preparing a radiotherapy treatment plan for a radiotherapy system, comprising:
acquiring a first image of a region of a patient including a target volume;
based on said first image, computing an initial treatment plan which, when implemented by the radiotherapy system, is expected to deliver a clinically acceptable dose to the patient;
for each of a plurality of hypothetical spatial variations of the target volume, determining a variation of the initial treatment plan consequent on the spatial variation;
computing the dose delivered by the thus-varied treatment plan to verify whether it would deliver a clinically acceptable dose to the patient and thereby determining a range of acceptable spatial variations;
acquiring a second image of the target region and determining an actual spatial variation of the target region relative to the first image;
checking whether the actual spatial variation is within the range of acceptable spatial variations and, if so, varying the initial treatment plan consequent on the actual spatial variation.

2. The method as claimed in claim 1, further comprising:
measuring the dose delivered by the initial treatment plan in the absence of the patient; and
verifying whether said initial treatment plan delivers a clinically acceptable dose to the patient.

3. The method as claimed in claim 1, further comprising:
measuring the dose delivered by the thus-varied treatment plan in the absence of the patient; and
verifying whether said thus-varied treatment plan delivers an acceptable dose to the patient.

4. The method as claimed in claim 1, wherein said spatial variations include only translation and rotation of the target region.

5. The method as claimed in claim 1, wherein said spatial variations include one or more of translation, rotation, scaling and deformation of the target region.

6. The method as claimed in claim 1, wherein for each of the plurality of hypothetical spatial variations of the target volume, the variation of the initial treatment plan is calculated using an algorithm, and wherein, for the actual spatial variation of the target volume, the initial treatment plan is varied according to said algorithm.

7. The method as claimed in claim 5, wherein said algorithm applies a perturbation of the initial treatment plan.

8. The method as claimed in claim 5, wherein said algorithm is selected from a plurality of available algorithms.

9. The method as claimed in claim 8, wherein said algorithm is selected based on the nature of the spatial variations and the nature of the target volume.

10. The method as claimed in claim 1, wherein said plurality of hypothetical spatial variations comprises, for each type of spatial variation, a regular sequence of variations of differing magnitude.

11. The method as claimed in claim 1, wherein said plurality of hypothetical spatial variations comprises, for each type of spatial variation, a stochastic sequence of variations of differing magnitude.

12. A treatment planning apparatus for radiotherapy, comprising:
a computing element;
a digital data storage;
the digital data storage being configured to retain a first image of a region of a patient including a target volume;
a stored computer program, adapted to direct the computing element to:
based on said first image, compute an initial treatment plan which when implemented by the radiotherapy system is expected to deliver a clinically acceptable dose to the patient;
determining a variation of the initial treatment plan consequent on the spatial variation;
compute the dose delivered by the thus-varied treatment plan to verify whether it would deliver a clinically acceptable dose to the patient repeat steps 0. and 0. for each of a plurality of hypothetical spatial variations of the target volume, thereby determine a range of acceptable spatial variations;
the digital data storage being further configured to retain a second image of the target region;
the stored computer program being further adapted to direct the computing element to:
determine an actual spatial variation of the target region relative to the first image;
check whether the actual spatial variation is within the range of acceptable spatial variations;
if so, vary the initial treatment plan consequent on the actual spatial variation.

13. The treatment planning apparatus of claim 12, wherein said spatial variations include only translation and rotation of the target region.

14. The treatment planning apparatus of claim 12, wherein said spatial variations include one or more of translation, rotation, scaling and deformation of the target region.

15. The treatment planning apparatus of claim 12, wherein for each of the plurality of hypothetical spatial variations of the target volume, the stored computer program is adapted to direct the computing element to vary the initial treatment plan using an algorithm, and wherein, for the actual spatial variation of the target volume, the stored computer program is adapted to direct the computing element to vary the initial treatment plan according to said algorithm.

16. The treatment planning apparatus of claim 15, wherein said algorithm is adapted to apply a perturbation of the initial treatment plan.

17. The treatment planning apparatus of claim 15, wherein the stored computer program selects said algorithm from a plurality of available algorithms.

18. The treatment planning apparatus of claim 17, wherein the stored computer program selects said algorithm based on criteria including the nature of the spatial variations and the nature of the target volume.

19. The treatment planning apparatus of claim 12, wherein said plurality of hypothetical spatial variations comprises, for each type of spatial variation, a regular sequence of variations of differing magnitude.

20. The treatment planning apparatus of claim 12, wherein said plurality of hypothetical spatial variations comprises, for each type of spatial variation, a stochastic sequence of variations of differing magnitude.
